(19) United States
(12) Patent Application Publication
(10) Pub. No.: US 2015/0305908 A1
(43) Pub. Date: Oct. 29, 2015

(54) KNEE BRACE

(71) Applicant: Shaun Spade, Geneva, OH (US)

(72) Inventor: Shaun Spade, Geneva, OH (US)

(21) Appl. No.: 14/263,023

(22) Filed: Apr. 28, 2014

(51) Int. Cl. A61F 5/01

(52) U.S. Cl. CPC A61F 5/0123

(57) ABSTRACT

A knee brace and method for supporting osteokinematic joint movement. The knee brace includes a tubular sleeve adapted to enwrap a user's knee and extend around portions of the user's upper leg and lower leg adjacent to the knee. Further, an elongated strapping component is secured to the tubular sleeve, and adapted to wrap about the tubular sleeve in a spiral configuration. The elongated strapping component is constructed of an elastic material that provides support for stabilizing the user's knee.

### TECHNICAL FIELD OF THE INVENTION

The present invention relates to a knee brace and more particularly to a knee brace that offers osteokinematic joint movement between the fibula/tibia and the femur during knee flexion and extension.

### BACKGROUND OF THE INVENTION

Knee impairments account for almost half of the musculoskeletal impairments in the United States. The knee is the largest joint in the body, and its exposed position makes it vulnerable to injury during activities. Further, the knee is acknowledged as one of the weakest joints in the body, and serves as the articulating joint between the thigh and calf muscle groups. The knee is held together primarily by small but powerful ligaments. Knee instability arising out of cartilage damage, ligament strain and other causes is relatively commonplace since the knee joint is subjected to significant loads during the course of almost any kind of physical activity requiring the use of the legs. Trauma to the knee is a very common orthopedic injury. Examples of knee injuries include bone fractures and breaks, ligament stretches and tears, twisting beyond the knee's allowable range of motion, injuries to the muscles and tendons surrounding the joint, and inflammation of the bursa. Knee impairments affect a person's ability to walk, crouch, climb and otherwise move freely. Additionally, knee impairments can be extremely debilitating and painful. Knee injuries typically require rest, surgery, rehabilitation, or a combination thereof.

Knee braces are commonly utilized to alleviate pain, provide stability, increased mobility, and reduced healing time after injury, medical procedure or ailment of the knee. Knee braces may be rigid or semi-rigid, providing medial and lateral knee stability to protect the medial and lateral collateral ligaments, meniscus and joint cartilage. Adjustable hinges may be included to provide protection of the ACL and PCL ligaments. Flexion and extension stops may be included to control knee joint range of motion. Additionally, by wrapping tightly around the leg the knee brace may provide compressive therapy. Knee braces alleviate pain and allow damaged tissue to properly heal and rehabilitate.

### SUMMARY OF THE INVENTION

According to an embodiment of the present invention, there is disclosed a knee brace that supports osteokinematic joint movement. The knee brace includes a tubular sleeve adapted to enwrap a user's knee and extend around portions of the user's upper leg and lower leg adjacent to the knee. Further, an elongated strapping component is secured to the tubular sleeve, and adapted to wrap about the tubular sleeve in a spiral configuration. The elongated strapping component is constructed of an elastic material that provides support for stabilizing the user's knee.

According to another embodiment of the present invention, there is disclosed a method of supporting osteokinematic joint movement in a user's knee with a knee brace. The method includes enwrapping a tubular sleeve about the user's knee whereby the tubular sleeve extends around portions of the user's upper leg and lower leg adjacent to the knee. The method further includes wrapping an elongated strapping component constructed of an elastic material about the tubular sleeve in a spiral configuration for stabilizing the knee.

According to a further embodiment of the present invention, there is disclosed an elbow brace that supports osteokinematic joint movement. The elbow brace includes a tubular sleeve adapted to enwrap a user's elbow and extend around portions of the user's upper arm and lower arm adjacent to the elbow. Further, an elongated strapping component is secured to the tubular sleeve, and adapted to wrap about the tubular sleeve in a spiral configuration. The elongated strapping component is constructed of an elastic material that provides support for stabilizing the user's elbow.

According to a further embodiment of the present invention, there is disclosed a method of supporting osteokinematic joint movement in a user's elbow with a elbow brace. The method includes enwrapping a tubular sleeve about the user's elbow whereby the tubular sleeve extends around portions of the user's upper arm and lower arm adjacent to the elbow. The method further includes wrapping an elongated strapping component constructed of an elastic material about the tubular sleeve in a spiral configuration for stabilizing the user's elbow.

### BRIEF DESCRIPTION OF THE DRAWINGS

The structure, operation, and advantages of the present invention will become further apparent upon consideration of the following description taken in conjunction with the accompanying figures (FIGs.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a front, three-dimensional view of an improved knee brace, in accordance with the present invention.

FIG. 2 is a front, three-dimensional view of a first position of a user applying the improved knee brace, in accordance with the present invention.

FIG. 3 is a front, three-dimensional view of a second position of a user applying the improved knee brace, in accordance with the present invention.

FIG. 4 is a front, three-dimensional view of a third position of a user applying the improved knee brace, in accordance with the present invention.

FIG. 5 is a front, three-dimensional view of a fourth position of a user applying the improved knee brace, in accordance with the present invention.

FIG. 6 is a front, three-dimensional view of an alternative embodiment of an improved elbow brace, in accordance with the present invention.

FIG. 7 is a front, three-dimensional view of an alternative embodiment of an improved elbow brace, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

In the drawings accompanying the description that follows, often both reference numerals and legends (labels, text descriptions) will be used to identify elements. If legends are provided, they are intended merely as an aid to the reader, and should not in any way be interpreted as limiting.

The objective of the improved knee brace 10 is to provide a knee brace that offers support for osteokinematic joint movement, i.e., the basic knee movements, which include extension, flexion, adduction, abduction, and rotation correction, between the fibula/tibia and femur during knee flexion and extension. Many people with knee pain report pain with knee flexion and extension during gait, especially during the weight bearing phase of the gait. Taping the knee, preferably with sports tape, has been found to be effective for alleviating these symptoms including preventing knee injuries, aiding the healing of knee injuries, reducing knee pain, and just increasing support in the knee. However, it is very difficult for patients to apply sports tape to themselves, and traditional elastic braces don't support the knee from stress, or address the osteokinematic movement of the knee. Current elastic bracing systems are for either limiting motion or unloading a joint or soft tissue, but do not address the osteokinematic joint rotation that occurs during normal joint movement of the knee to decrease pain from impingement or faulty mechanics. Further, both internal tibia rotation during flexion of the knee and external rotation during knee extension are not addressed with the current elastic bracing techniques.

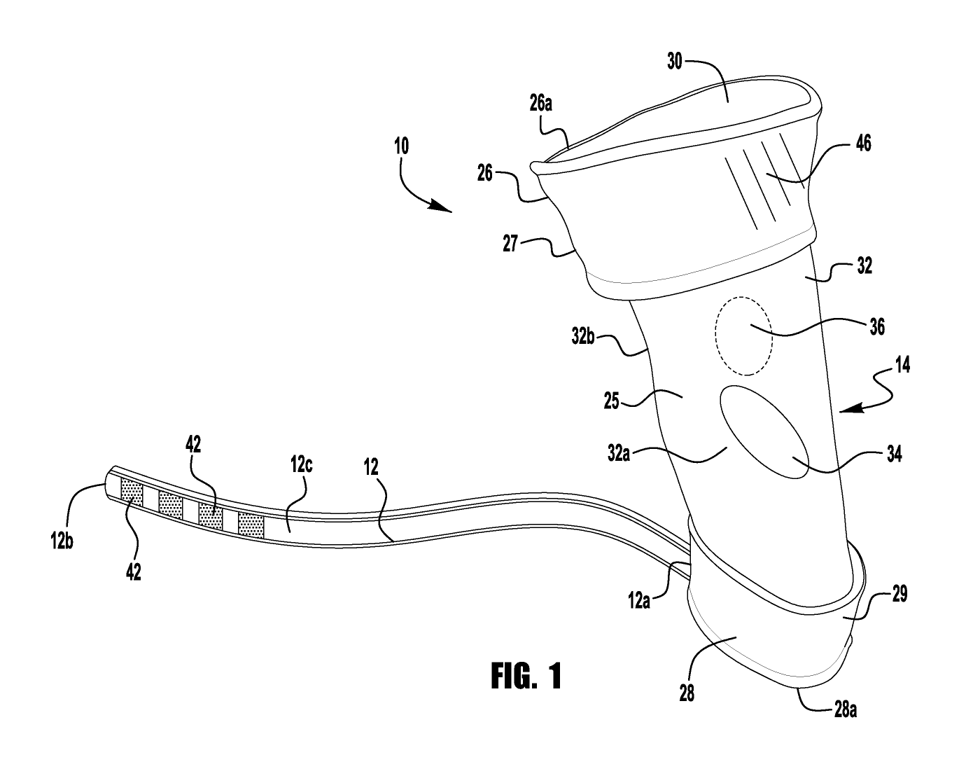

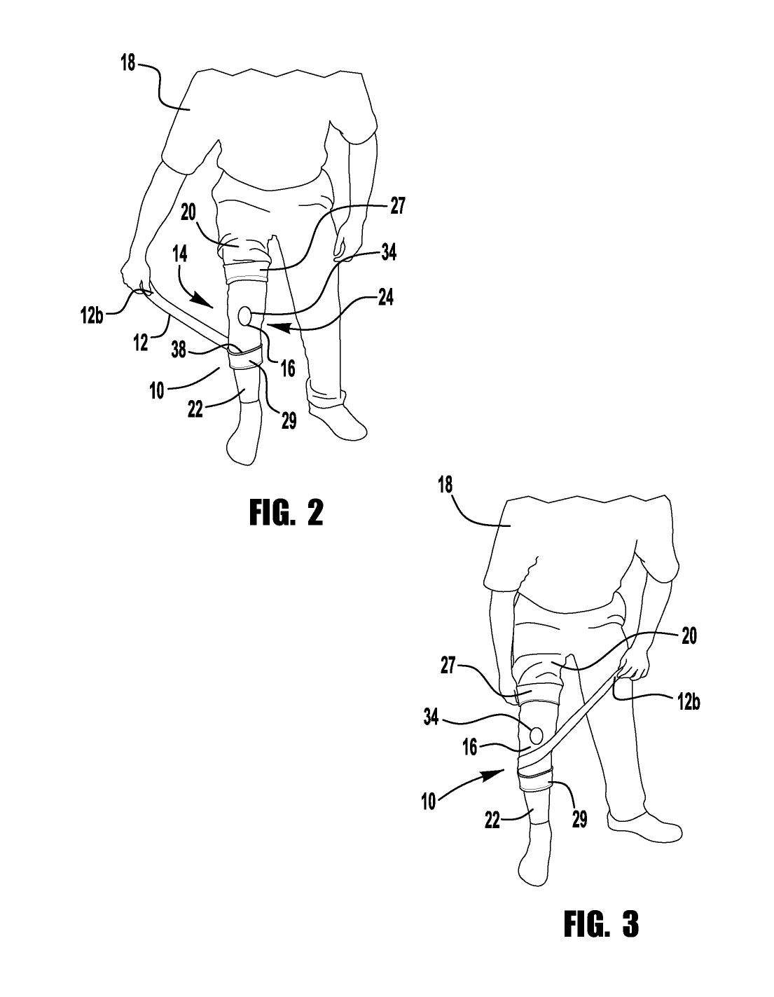

The improved knee brace 10, shown in FIG. 1, addresses joint movements similarly to the effective taping techniques currently used to alleviate pain. The improved knee brace 10 comprises a tubular sleeve 14, and an associated elongated strapping component 12 that is secured to and/or integral with the tubular sleeve 14. As seen in FIG. 2, and discussed in more detail hereinafter, the tubular sleeve 14 of the knee brace 10 is initially pulled up over the user's foot and lower leg until it is positioned around the user's knee 16 with portions of the tubular sleeve extending around adjacent portions of the upper and lower leg 20 and 22, respectively. As illustrated, the tubular sleeve 14 substantially enwraps the knee 16 and the adjacent portions of the upper and lower leg 20 and 22. For purposes of illustration, the improved knee brace 10 is configured to be worn on the right leg 24 of the user 18 for stabilizing the right knee 16. However, it is readily apparent from the description herein that the knee brace 10 can readily be adapted for wearing on the left leg of a user for stabilizing the left knee.

As seen in FIG. 1, the tubular sleeve 14 is comprised of a specially cut pattern of composite stretchable elastic material, such as neoprene, which has a tubular shape so that when it is disposed about the knee 16 of the user 18, it is snug fitting, provides comfortable uniform compression around the knee and has the ability to grab the user's skin to allow for needed joint internal rotation. Tubular sleeve 14 has a first end section 26 with an entrance opening 26a, an intermediate section 25, and a second end section 28 with an exit opening 28a through which the adjacent portions of the user's upper and lower leg 20 and 22, respectively, extend when the intermediate section is positioned about the user's knee. As illustrated, it is within the terms of the embodiment that there are additional gripping strips 27 and 29 of an elastic or stretchable material are disposed about the first end section 26 and the second end section 28, respectively, to provide additional compressive gripping support so that the tubular sleeve remains in place on the user 18. The material of the tubular sleeve 14 must be compressive and have the ability to grab the user 18 to allow for needed internal joint rotation.

In one embodiment, the interior layer 30 of tubular sleeve 14 is made of elastic or stretchable material, such as neoprene, configured to snugly embrace the skin about the knee 16. Further, the tubular sleeve 14 can have an exterior layer 32 adhered or bonded to the interior layer 30 and made of stretchable material.

Tubular sleeve 14 can have a first circular opening 34 through the intermediate section 25 formed substantially in the anterior of the intermediate section 25 of the tubular sleeve. The opening 34 may be approximately 2 to about 3 inches in diameter and is positioned so as to be aligned with the patella region of the user 18 when the knee brace 10 is worn. The opening 34 relieves pressure against the patella region, prevents abrasion of the skin during vigorous activity of the user 18, and increases the flexibility of the knee brace 10 at the patella. The opening 34 is positioned to lie directly over the knee 18 to allow the knee to protrude as not to create any direct pressure downward on the knee while simultaneously cupping all sides of the kneecap.

Further, tubular sleeve 14 can have a second circular opening 36 formed substantially in the posterior of the intermediate section 25 of the tubular sleeve. The opening 36 may be approximately 2 to about 3 inches in diameter and positioned so as to be aligned with the popliteal region of the user 18 when the knee brace 10 is worn. The opening 36 is designed to be disposed behind the knee 16 to allow for non-restrictive movement of the user 18.

While the tubular sleeve 14 is shown and described with a first circular opening 34 formed substantially in the anterior of the intermediate section 25 of the tubular sleeve and a second circular opening 36 formed substantially in the posterior of the intermediate section 25 of the tubular sleeve, it is within the scope of the preferred embodiment to form the tubular sleeve without either both of the first and second openings or alternatively without one of either the first or second openings.

The elongated strapping component 12 of the knee brace 10 is attached at a first end 12a to the tubular sleeve 14 adjacent to the exit opening 28a so that the first end 12a of the elongated strapping component 12 is located on the mid-distal calf region 38 of the user when the knee brace is worn by a user.

Typically, the end 12a of the elongated strapping component 12 is permanently affixed, such as by sewing, to the exterior layer of the tubular sleeve 14 or to the additional gripping strip 29 near the exit opening 28a. However it is within the terms of the embodiment that end 12a of strapping component 12 be releasably secured to the exterior surface of the tubular sleeve 14 or to the additional gripping strip 29 exterior region, such as with a Velcro® fastener. The strapping component 12 may be any appropriate dimensions, such as a length of approximately 16 to about 20 inches and a width of approximately 1.5 to about 2 inches.

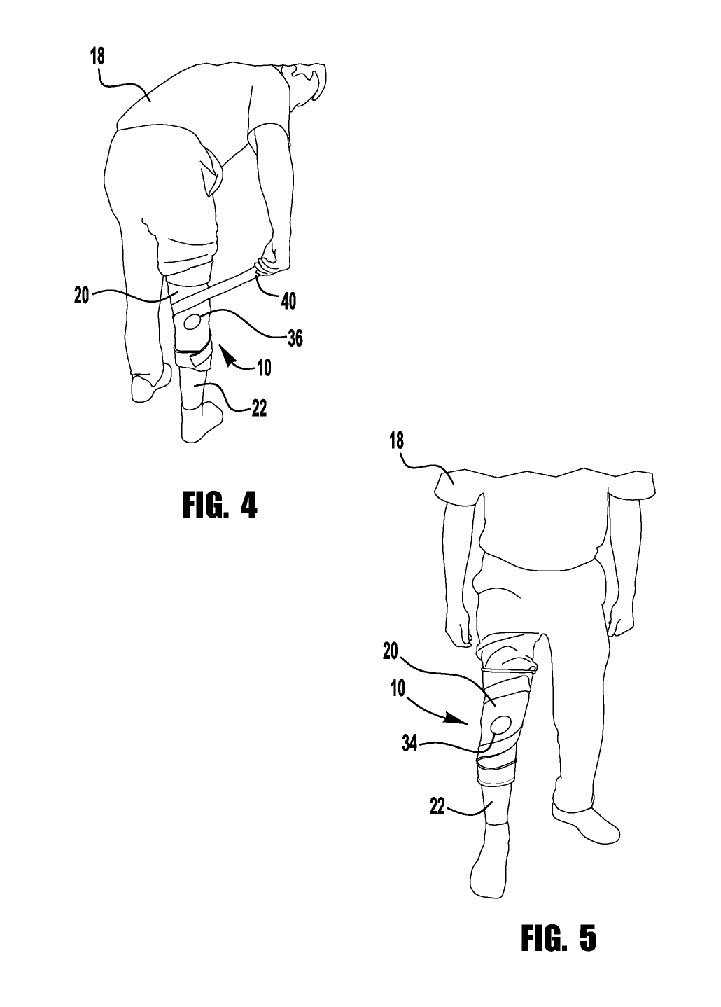

The strapping component 12 is designed to wrap about the tubular sleeve 14, once the tubular sleeve is in place on the user, and around the user's knee 16 in a generally spiral configuration similar to the application of sport tape. The strapping component 12 can be constructed of elastic material, such as a neoprene material. The elastic properties of the strapping component 12 allows the strapping component to replicate tape. That is by positioning the first end 12a of the strapping component 12 on the outer surface of the tubular sleeve 14 above the mid posterior tibia of the user 18 as shown in FIG. 2, the second end 12b of the strapping component is held by the user in order to wind the strapping component in a spiral configuration about the tubular sleeve. The strapping component 12 is wrapped about the tubular sleeve 14 to first circle anteriorly above the tibial tuberosity as shown in FIG. 3, then above the medial knee joint line, continuing above the posterior distal femur as shown in FIG. 4 and finally wrapping anteriorly to above the anterior femur as shown in FIG. 5. The strapping component 12 is designed to replicate the taping procedure and provide the necessary tibial internal rotation and femoral external rotation to address the osteokinematic joint rotation that occurs during normal joint movement of the knee. The combination of the tubular sleeve 14 and the strapping component 12 enables a user 18 to independently put on the tubular sleeve 10 by first positioning the tubular sleeve 14 of the knee brace 10 around the user's knee 16 and then wrapping the strapping component 12 about the tubular sleeve in a spiral configuration so that the elasticity of the strapping component 12 provides support for stabilizing the knee in a manner similar to the current supportive taping technique.

The free second end 12b of the elongated strapping component 12 has a means for connecting it to the tubular sleeve 14. As illustrated, portions of a hook material 42, such as Velcro®, are sewn or adhered to an interior surface 12c adjacent to the free second end 12b for mating engagement with a loop material 46, such as Velcro®, disposed on the exterior surface of the first end section 26 or the additional gripping strip 27 of tubular sleeve 14. After the user 18 puts on the tubular sleeve 14 about knee 16, the strapping component 12 is wrapped about the tubular sleeve in a generally spiral configuration, as discussed hereinbefore, in a manner similar to tape. After the strapping component 12 is sufficiently tightened, the hook material 42 at the second end 12b of the strapping component is pressed against the loop material 46 disposed on the exterior surface of the first end section 26 or the additional gripping strip 27 of tubular sleeve 14 to provide the desired compression of the tubular sleeve 14 about the knee of the user.

FIGS. 2, 3, 4, and 5 illustrate the steps of a user 18 applying the knee brace 10. A properly sized knee brace is initially selected based on the size of the user. The size of the selected knee brace 10 is based on the circumferential measurements of the user's leg 18 taken about 6 inches proximal (towards the hip) to mid-patella, at mid-patella and about 6 inches distal (towards the calf) to mid-patella. First, while sitting or standing, the user steps into the tubular sleeve 14 of the knee brace 10 and pulls it up so that the circular opening 34 through the intermediate section 25 is located directly over the patella. Next, the user grasps the end 12b of strapping component 12. Next, as seen in FIG. 2, the user 18 stands (seated will also work) with the knee 16 slightly bent, typically at an angle of approximately 10° to about 15°, and pulls the end 12b of the strapping component 12 about the outside surface of the tubular sleeve 14 in a direction away from his knee 16. Next, as shown in FIG. 3, the user 18 pulls the strapping component 12 around the outside aspect of the upper leg 20 (tibia/fibula) and across the intermediate section 25 of the tubular sleeve 14 just above the first circular opening 34. Then, as seen in FIG. 4, the user 18 continues to wrap the strapping component 12 behind the thigh (femur), around the outside (lateral) side of the femur and above the knee. Continuing in FIG. 5, the user 18 wraps the strapping component 12 about the outside aspect of the upper leg 20 until the hook material 42 of the interior region 44 of the free second end 40 can attach to the loop material 46.

It should be noted that the strapping component 12 of the knee brace 10 provides and sustains tibial/fibula internal rotation with concomitant femoral external rotation. To best achieve this, the user 18 is positioned such that the knee 16 is flexed approximately 10° to 25°, with the foot, of the affected knee, elevated, such as on a step. Then, the user 18 is positioned such that the foot is turned inward while rotating his trunk outward towards the affected knee 16. For example, if the right leg requires the knee brace, the user rotates his trunk to the right. The former positioning has been found to be the most effective body positioning for putting on a knee brace 10, as described herein.

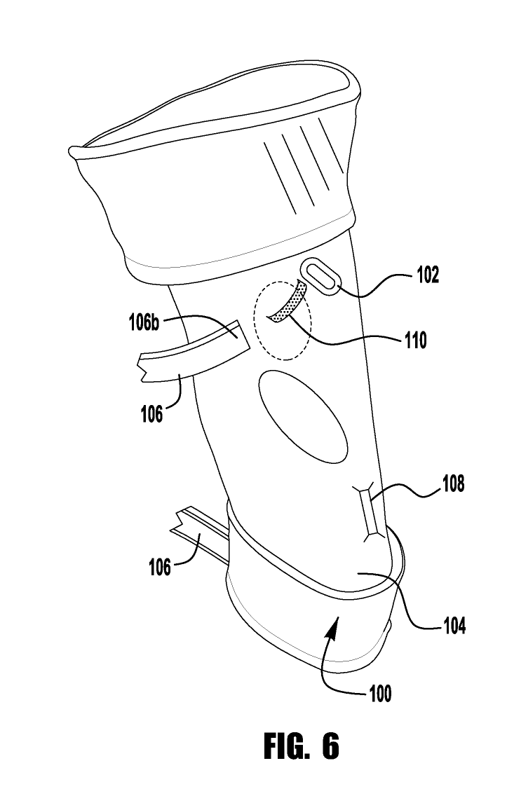

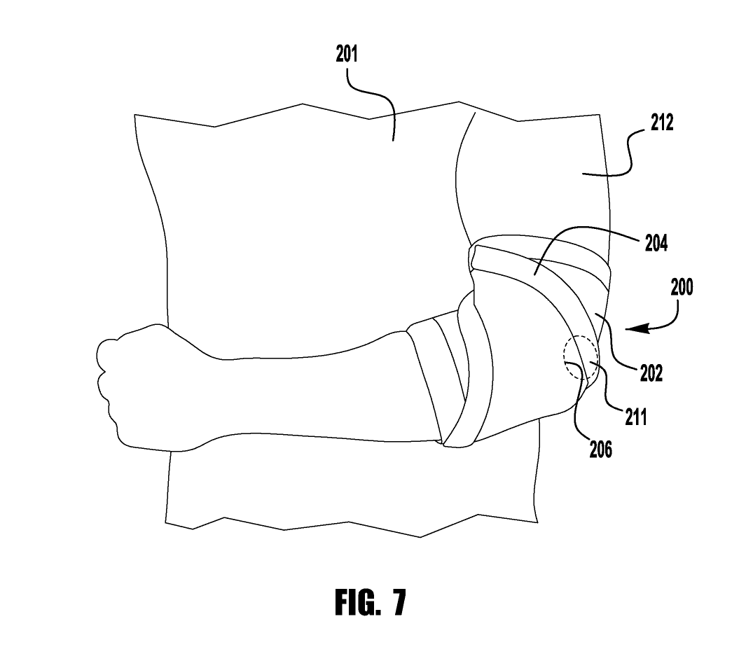

FIG. 6 illustrates an alternative embodiment of the knee brace 100 (compare knee brace 10). Improved knee brace 100 is identical to the improved knee brace 10 in appearance and function with identical components with several additions. In the alternative embodiment, knee brace 100 includes a strip 110 of looped Velcro attached to the tubular sleeve 104 (compare tubular sleeve 14) of the brace 100 to connect to hooked Velcro (not shown) located at the end 106b of the strapping component 106. In the alternative embodiment, knee brace 100 can also include a metal or plastic loop 102 secured at a medial aspect of the tubular sleeve 104 (compare tubular sleeve 14) of the brace 100 through which the strapping component 106 is passed to allow for greater pull on the strapping component 106 before it is secured with the hooked material at the end 106b to the strip 110 of looped Velcro attached to the tubular sleeve 104. In addition, a metal or plastic hooked end 108 is placed on the tubular sleeve 104 to allow the strapping component 106 to pass there through and hold it in place with respect to the tubular sleeve 104.

FIG. 7 illustrates an alternative embodiment of an improved elbow brace 200 (comparable to the improved knee brace 10) that is worn by a user 201. The improved elbow brace 200 essentially operates in a manner similar to the improved knee brace 10, with the obvious exception that the improved elbow brace is designed to be worn about the elbow. The improved elbow brace 200 addresses osteokinematic joint movements similarly to the effective taping techniques currently used to alleviate pain. During active elbow flexion, the ulna and radius internally rotate on the humerus, and conversely during active elbow extension, the ulna and radius externally rotation on the humerus. Common injuries to the elbow include tennis elbow, golfer's elbow, and biceps strain. The improved elbow brace 200 is designed to assist in radius and ulna internal rotation with the humerus as in traditional taping methods.

The improved elbow brace 200 comprises a tubular sleeve 202 constructed of an elastic, stretchable material, and an associated elongated strapping component 204 that is secured to and/or integral with the tubular sleeve 202. The tubular sleeve 202 of the elbow brace 200 is initially pulled up over the user's 201 hand and lower arm until it is positioned around the user's elbow 206 with portions of the tubular sleeve extending around adjacent portions of the upper and lower arm 208 and 210, respectively. As illustrated, the tubular sleeve 202 substantially encloses the elbow 206 and the adjacent portions of the upper and lower arm 208 and 210. respectively. There may be an anterior opening 211 and posterior opening (not shown) at the elbow joint. For purposes of illustration, the improved elbow brace 200 is configured to be worn on the left arm 212 of the user 201 for stabilizing the left elbow 206. However, it is readily apparent from the description herein that the elbow brace 200 can readily be adapted for wearing on the right arm of a user for stabilizing the right elbow.

The strapping component 204 is constructed of an elastic, stretchable material (comparable to the improved knee brace 10) and designed to wrap about the tubular sleeve 202, once the tubular sleeve is in place on the user 201, and around the user's elbow 206 in a generally spiral configuration similar to the application of sport tape. The elastic properties of the strapping component 204 allows the strapping component to replicate tape. The strapping component 204 essentially operates in a manner identical to the strapping component 12 of the knee brace 10. The strapping component 204 is positioned on the outer surface of the tubular sleeve 202 distal to the elbow 206, and is wrapped about the outer surface of the tubular sleeve laterally, then anteriorly across the user's 201 forearm, continuing medially across the medial elbow joint and then laterally along the posterior humerus and finishing anteriorly across the bicep.

According to an embodiment of the invention, a method of supporting osteokinematic joint movement in a user's elbow 206 with an elbow brace 200. The method includes enwrapping a tubular sleeve 202 about the user's elbow 206 whereby the tubular sleeve 202 extends around portions of the user's upper arm and lower arm adjacent to the elbow. Next, an elongated strapping component 204 constructed of an elastic material is wrapped about the tubular sleeve 202 in a spiral configuration for stabilizing the user's elbow 206.

Initially, the user pulls a first end of the strapping component 204 about an outside surface of the tubular sleeve 202 in a direction away from the user's elbow 206. Then the strapping component 204 is pulled around the outside aspect of the upper arm and across an intermediate section the tubular sleeve 202. Continuing, the strapping component 204 is wrapped behind the upper arm and across an intermediate section the tubular sleeve 202. Further, the strapping component 204 is wrapped behind the user's upper arm and above the elbow 206. Next, a second free end of the strapping component 204 is attached to the elbow brace 200.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described components (assemblies, devices, etc.) the terms (including a reference to a “means”) used to describe such components are intended to correspond, unless otherwise indicated, to any component which performs the specified function of the described component (i.e., that is functionally equivalent), even though not structurally equivalent to the disclosed structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. A knee brace that supports osteokinematic joint movement, comprising:
a tubular sleeve adapted to enwrap a user's knee and extend around portions of the user's upper leg and lower leg adjacent to the knee; and
an elongated strapping component being secured to the tubular sleeve, and adapted to wrap about the tubular sleeve in a spiral configuration, the elongated strapping component constructed of an elastic material that provides support for stabilizing the user's knee.

2. The knee brace of claim 1 wherein the tubular sleeve is comprised of a stretchable material configured to snugly embrace the user's knee.

3. The knee brace of claim 2 wherein the tubular sleeve has a first end section with an entrance opening, an intermediate section, and a second end section with an exit opening through which the adjacent portions of the user's upper and lower leg, respectively, extend when the intermediate section is positioned about the user's knee.

4. The knee brace of claim 3 wherein additional gripping strips of a stretchable material are disposed about the first end section and the second end section, respectively, to provide additional compressive gripping support.

5. The knee brace of claim 3 wherein a first circular opening is formed substantially in an anterior of the intermediate section of the tubular sleeve and positioned so as to lie directly over the user's knee.

6. The knee brace of claim 5 wherein a second circular opening is formed substantially in a posterior of the intermediate section of the tubular sleeve so as to be disposed behind the user's knee.

7. The knee brace of claim 3 wherein the tubular sleeve is comprised of:
an interior layer made of stretchable material configured to snugly embrace the user's knee; and
an exterior layer made of stretchable material bonded to the interior layer.

8. The knee brace of claim 3 wherein the strapping component of the improved knee brace is attached at a first end to the tubular sleeve adjacent to the exit opening so that the one end is located on a mid-distal calf region of the user.

9. The knee brace of claim 8 wherein a second end of the strapping component has a means for connecting it to the tubular sleeve.

10. The knee brace of claim 9 wherein portions of hook material are adhered to an interior surface of the strapping component for mating engagement with loop material disposed on the first end section of the tubular sleeve.

11. A method of supporting osteokinematic joint movement in a user's knee with a knee brace, comprising:
enwrapping a tubular sleeve about the user's knee whereby the tubular sleeve extends around portions of the user's upper leg and lower leg adjacent to the knee; and
wrapping an elongated strapping component constructed of an elastic material about the tubular sleeve in a spiral configuration for stabilizing the knee.

12. The method of claim 11 further including:
pulling a first end of the strapping component about an outside surface of the tubular sleeve in a direction away from the user's knee;
pulling the strapping component around the outside aspect of the upper leg and across an intermediate section the tubular sleeve;
wrapping the strapping component behind the thigh and across an intermediate section the tubular sleeve;
wrapping the strapping component behind the user's thigh and above the knee; and
attaching a second free end of the strapping component to the knee brace.

13. The method of claim 12 further including:
selecting a properly sized knee brace based on the size of the user;
stepping into the knee brace and pulling it up so that a circular opening disposed within the brace is located directly over the user's patella; and
pulling the end of the strapping component around an outside aspect of the user's upper leg and across the knee brace just above the circular opening.

14. An elbow brace that supports osteokinematic joint movement, comprising:
a tubular sleeve adapted to enwrap a user's elbow and extend around portions of the user's upper arm and lower arm adjacent to the elbow; and
an elongated strapping component being secured to the tubular sleeve, and adapted to wrap about the tubular sleeve in a spiral configuration, the elongated strapping component constructed of an elastic material that provides support for stabilizing the user's elbow.

15. The elbow brace of claim 14 wherein the tubular sleeve is comprised of a stretchable material configured to snugly embrace the user's elbow.

16. The elbow brace of claim 15 wherein the tubular sleeve has a first end section with an entrance opening, an intermediate section, and a second end section with an exit opening through which the adjacent portions of the user's upper and lower leg, respectively, extend when the intermediate section is positioned about the user's elbow.

17. The elbow brace of claim 16 wherein additional gripping strips of a stretchable material are disposed about the first end section and the second end section, respectively, to provide additional compressive gripping support.

18. The elbow brace of claim 17 wherein portions of hook material are adhered to an interior surface of the strapping component for mating engagement with loop material disposed on the first end section of the tubular sleeve.

19. A method of supporting osteokinematic joint movement in a user's elbow with a elbow brace, comprising:
enwrapping a tubular sleeve about the user's elbow whereby the tubular sleeve extends around portions of the user's upper arm and lower arm adjacent to the elbow; and
wrapping an elongated strapping component constructed of an elastic material about the tubular sleeve in a spiral configuration for stabilizing the user's elbow.

20. The method of claim 19 further including:
pulling a first end of the strapping component about an outside surface of the tubular sleeve in a direction away from the user's elbow;
pulling the strapping component around the outside aspect of the upper arm and across an intermediate section the tubular sleeve;
wrapping the strapping component behind the upper arm and across an intermediate section the tubular sleeve;
wrapping the strapping component behind the user's upper arm and above the elbow; and attaching a second free end of the strapping component to the elbow brace.
